(19) United States
(12) Patent Application Publication
(10) Pub. No.: US 2025/0387043 A1
(43) Pub. Date: Dec. 25, 2025

(54) SYSTEM AND METHOD FOR MEASURING THE MECHANICAL IMPEDANCE OF THE RESPIRATORY SYSTEM

(71) Applicant: RESTECH S.R.L, Milan (IT)

(72) Inventors: Pasquale Pio POMPILIO, Milan (IT); Alessandro GOBBI, Bergamo (IT)

(21) Appl. No.: 19/108,027

(22) PCT Filed: Sep. 1, 2023
(86) PCT No.: PCT/EP2023/074066

§ 371 Date: Feb. 28, 2025

(30) Foreign Application Priority Data
Sep. 2, 2022 (IT) 102022000018084

(51) Int. Cl. A61B 5/085; A61B 5/00; A61B 5/087

(52) U.S. Cl. CPC A61B 5/085; A61B 5/0053; A61B 5/087; A61B 2560/0223

(57) ABSTRACT

A system for measuring the mechanical impedance of a patient's respiratory system during spontaneous respiratory activity characterised in that it comprises a fan, a motor driving said fan; said motor and said fan are placed inside a cavity; said cavity is divided into two sub-cavities separated from each other so as to prevent the passage of air, one containing the motor and the other containing the fan and comprising an initial end and a terminal end, both of which have access to the outside.

### TECHNICAL FIELD

The present invention relates to a system and method for measuring the mechanical impedance of the respiratory system during the spontaneous breathing activity of an individual (e.g. a patient) using the forced oscillation technique (FOT or oscillometry).

### STATE OF THE ART

Oscillometry is a method of measuring the mechanical properties of the airways and lungs based on analysis of the airflow resulting from the application of small-amplitude pressure stimuli oscillating at a frequency above that of spontaneous breathing. This method, first proposed in 1956 by the American physiologist Arthur Dubois, (Journal of Applied Physiology—May 1956, vol. 8 no. 6, 587-594), has aroused increasing interest from the clinical world in recent years as a potential new non-invasive and easy-to-use means for diagnosing and monitoring respiratory system dysfunctions.

This, together with the appearance on the market of new devices making use of the method, recently prompted the European Respiratory Society to publish a technical standards document to support the dissemination of such instruments in the clinical setting (Eur Respir J 2020; 55:1900753).

During an oscillometric measurement, the respiratory system is subjected to an oscillating mechanical stimulus of small amplitude produced by an external actuator. The difficulty of bringing about a subsequent movement of air through the respiratory system from the stimulus is quantified by calculating the mechanical impedance (Z), which is obtained from the complex ratio between pressure (Pm) and flow (V′m) measured at the entry to the airways in relation to any or all of the frequency components (f) of the stimulus:

$$Z ( f ) = \frac{Pm ( f )}{{\dot{V}}_{m} ( f )} = R ( f ) + j * X ( f )$$

Mechanical impedance is a complex number, the real part of which, called resistance (R(f)), is an indicator of the caliber of the airways and/or their patency, while the imaginary part, called reactance (X(f)), summarises the system's ability to store energy and is thus determined by both the elastic and the inertial properties of the respiratory system. Various oscillometric measurement systems characterised by the use of different stimulus generation systems have been reported in the scientific and technical literature.

As first used, the stimulus generator consisted of a cylinder coupled to a piston, the outlet from which was directly connected to the airway opening (nose or mouth), and a set of sensors for measuring flow and pressure (Journal of Applied Physiology—May 1956, vol. 8 no. 6, 587-594, U.S. Pat. No. 3,713,436—Filed Oct. 23, 1970). These bulky and complex systems could not be used for measurements during spontaneous breathing, but only during short periods of apnoea.

Subsequently, from the late 1960s, oscillometric measurement systems consisting of a loudspeaker generating the oscillations connected to a set of pressure and flow sensors directly in contact with the individual's airway opening and, in parallel, a high-inertance pathway consisting of a tube of sufficient diameter and length and/or a resistance began to be more widely used to allow measurement during spontaneous breathing (The Journal of Clinical Investigation—November 1975 vol. 56 1210-1230, U.S. Pat. No. 4,333,476, EP 1 551 293). However, the presence of the high inertance tube greatly increased the dead space in the respiratory system and required the use of an additional flow generator to refresh the air, increasing the size and complexity of the overall system.

Smaller setups using actuators to partly or wholly occlude the airway during spontaneous breathing have been constructed in order to cause pressure disturbances within the circuit, where the stimulation energy is generated by the respiratory muscles, as described in patents U.S. Pat. Nos. 4,220,161 and 6,066,101.

Although the devices belonging to the latter category are cheaper and less bulky, they do not work for low expiratory and inspiratory flows (e.g. at the end of inspiration and end of expiration) and are not suitable for measuring reactance.

A small and potentially portable respiratory impedance measurement system has been described in patent application ITBG20100042 by the proprietor of the present patent application. This system comprises a cavity through which the individual being measured breathes, and which houses a motor connected to a fan whose suitably controlled movement is capable of generating the pressure oscillations needed for stimulation of the respiratory system and the measurement of respiratory impedance.

The object of the present invention is to provide a system and method for mechanical measurement of the respiratory system that at least partly overcomes the disadvantages of existing systems or improves their performance.

### OBJECT OF THE INVENTION

The main object of the present invention is to provide a system and method for measuring respiratory impedance that reduces the disadvantages of the known art.

### SUMMARY OF THE INVENTION

In accordance with the present invention, this result has been achieved through the construction of a portable system for measuring the mechanical impedance of a patient's respiratory system during spontaneous breathing, comprising: a system, comprising a motor and a fan, for generating pressure stimuli, able to produce small variations in pressure and/or flow at the opening to the airways; a detection system able to measure the pressure and flow values produced by the stimulation and by the patient's spontaneous breathing activity; a microprocessor able to: control the system for generating pressure stimuli; receive the data measured by the detection system; calculate a measurement of the mechanical impedance of the patient's respiratory system based on the values measured by the detection system, characterised in that it comprises a first chamber containing the motor and a second chamber containing the fan, the first and second chambers being separate from each other so that there is no passage of air between the first and second chambers, the second chamber having a rear opening able to take in air from the external environment and a front opening able to be brought into contact with the patient and to receive the patient's inspiratory and expiratory flows.

The detection system preferably comprises at least one pressure sensor. According to a preferred embodiment, the detection system also comprises a flowmeter for measuring the flow produced by the patient's spontaneous breathing activity; alternatively, the flow produced by the patient's spontaneous breathing activity is estimated by the microprocessor on the basis of one or more of the following data: fan rotation speed, motor power consumption, pressure, air temperature and humidity.

In a preferred embodiment, the microprocessor is arranged to modulate the pressure stimuli so that, during an initial measurement stage, the rotation speed is reduced compared to a steady-state measurement stage. The fan speed in the initial stage can be controlled by the microprocessor to produce a pressure stimulus whose peak-to-peak amplitude increases linearly until a predefined target value is reached. According to a possible alternative embodiment, the rotation speed of the fan in the initial stage can be controlled by the microprocessor to produce a pressure stimulus whose peak-to-peak amplitude increases exponentially until reaching a predefined target value. According to a preferred embodiment, the rotation speed of the fan during the steady-state measurement stage (i.e. after the acclimatisation stage) is controlled by a closed-loop control system to produce a pressure stimulus whose peak-to-peak amplitude is kept constant and equal to a value determined on the basis of one or more of the patient's pressure, flow, impedance, resistance and reactance values measured during the initial measurement stage. According to another aspect of the present invention, a method is provided for operating a portable system as defined above, comprising the following steps: generating pressure stimuli, producing small pressure variations; measuring the pressure and flow values produced by the stimulation and the patient's spontaneous breathing activity; receiving from the microprocessor the data measured by the sensing system; calculating a measurement of the mechanical impedance of the patient's respiratory system based on the values measured by the sensing system.

A computer program which implements the method described above when executed by a microprocessor is also provided.

According to a further aspect of the present invention, there is provided a kit for measuring the mechanical impedance of a patient's respiratory system during spontaneous breathing, comprising: the system described above; a test and calibration device, comprising a hollow conduit having two substantially truncated conical opposite ends whose smaller cross-section converges inwardly, the two truncated conical ends being joined together by a substantially cylindrical central portion; the test device having a known and predetermined impedance value.

The present invention allows for the development of compact portable devices that can be used on different individuals, are easy to clean and maximise patient comfort during measurement.

Among the advantages obtained from devices made according to embodiments of the present invention, we would point out:
the separation between the chamber containing the motor and that containing the fan enables the motor to be isolated from the patient's inhaled and exhaled air. This prevents the inhalation of dust accidentally generated during motor operation and prevents moisture and any saliva produced during breathing from damaging the motor's circuits and electrical connections;
said separation also makes it possible to replace only the respiratory chamber and the fan in it if contamination occurs and allows for the possibility of using the device on different patients without having to replace the whole device;
the generation of progressively increasing stimulation during the acclimatisation stage of measurement, as provided for in one optional embodiment of the present invention, helps the individual to become accustomed to the presence of the stimulus oscillations, increasing the acceptability of the test;
the generation of a stimulus whose amplitude is adjusted on the basis of the amplitude of the resulting flow oscillations, provided by a possible embodiment of the present invention, makes it possible to optimise the signal-to-noise ratio and make use of pressure stimuli of reduced amplitude in individuals having low respiratory impedance, increasing comfort during measurement.

### BRIEF DESCRIPTION OF FIGURES

These and further advantages, purposes and features of the present invention will be better understood by any person skilled in the art from the description below and the accompanying drawing, which relate to examples of embodiments of an illustrative nature, but which are not to be understood in a limiting sense, in which

FIG. 1 is a diagrammatical illustration of a system for measuring the mechanical impedance of the respiratory system in accordance with a preferred embodiment of the present invention;

FIG. 2 diagrammatically depicts the pressure pattern at the patient's mouth during the acclimatisation and measurement stages according to a preferred embodiment of the present invention;

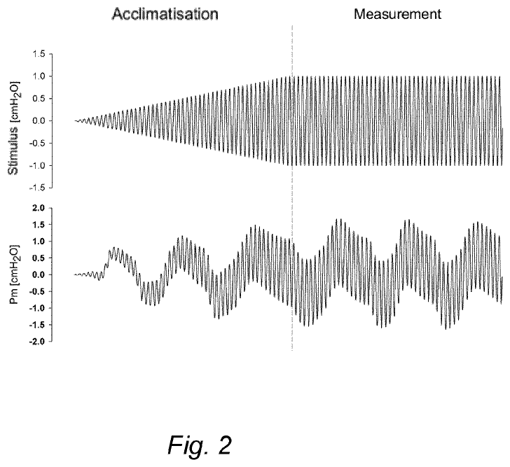

FIG. 3 illustrates a mechanical device for calibrating a system according to the present invention and for automatically checking correct system functioning.

### DETAILED DESCRIPTION

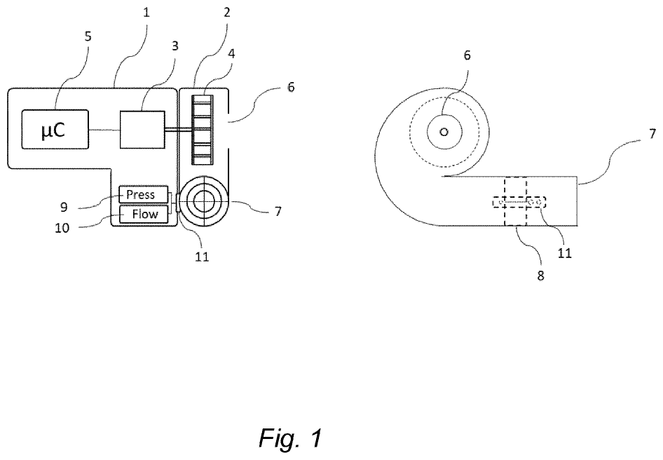

With reference to the appended figures, in particular FIG. 1, a system for measuring the mechanical impedance of the respiratory system according to a preferred embodiment of the present invention comprises a motor 3 connected to a centrifugal fan 4.

Motor 3 is located within a first chamber 1 which is not in communication with the patient's inhaled and exhaled air. Fan 4 is located within a second chamber 2 (“respiratory chamber”), having an initial end 7 and a terminal end 6, both with openings to the outside. The first and second chambers are placed in communication with each other so as to transmit mechanical movement from the motor to the fan, but not to allow air to pass between the two chambers.

Chamber 1 housing the motor and respiratory chamber 2 may also be contained in the same chamber (or cavity) from which two separate subchambers (or subcavities) are obtained, for example by means of a partition wall which allows mechanical communication (e.g. the motor shaft turning the fan), but which prevents the passage of air, so that the air inhaled by the user (patient) does not contain any harmful dust produced by the motor. This is one of the advantages provided by the systems according to the present invention over, for example, the system described in patent application ITBG20100042.

Initial end 7 is designed to be connected to a mouthpiece or other interface with the patient and is preferably about 2-4 cm in diameter.

In an alternative embodiment, chamber 2 may house a fan 4 of the axial type.

The distance travelled by the air inside the chamber is less than 25 cm, preferably about 15 cm, so that the device is easy to transport.

Where the volume of respiratory chamber 2 is greater than 50 ml, it preferably includes one or more vent holes, located approximately halfway between initial end 7 and the sampling ports for pressure and flow signals 11, which are needed to ensure outward diffusion of the exhaled air.

In accordance with one possible embodiment, the respiratory chamber includes a flowmeter 8 comprising a resistive element capable of producing a pressure drop able to produce a known change in pressure and function of the airflow through it. In an alternative embodiment, flow measurement at the airway opening is without the resistive element and can be performed using ultrasonic or hot-wire type sensors.

In a further embodiment, the flow may be estimated from the rotation speed and/or electricity consumption and/or pressure values measured within chamber 2 and in the environment.

In accordance with a possible embodiment, chamber 1 also comprises a pressure sensor 9 (Pm) and a flow sensor 10 (V′_m), placed in communication with respiratory chamber 2 via pneumatic connections 11.

Associated with chamber 1 is a microprocessor-based processing and control system 5, powered by the mains or batteries, which receives the signals from sensors 9 and 10, stores them in its memory, and performs the necessary processing to calculate the mechanical impedance of the respiratory system. The processing system also includes motor drive circuit 3, and a module for communication with the outside world for taking measurements and sending/receiving commands to/from the microprocessor. According to one embodiment, processing and control device 5, which manages the measurements taken, comprises a memory and electronic interfaces for data retrieval. In another embodiment, it comprises a data processing system in addition to the memory and thus directly provides data previously processed.

In another embodiment, the device includes a system for sending wireless data.

In another embodiment, the device includes a system for sending data to external processing and storage systems via the Internet.

In another embodiment, the device may include a sensor for measuring blood saturation and/or heart rate.

In another embodiment, the device may include a display showing measured values and system information.

In another embodiment, the device may include input systems such as buttons or touch screens for entering patient information and changing system settings.

The rotation speed of motor 3 is controlled by the microprocessor to force outside air into respiratory chamber 2 to produce pressure variations of maximum peak-to-peak amplitude of 5 cm H₂O, of a predefined shape, typically sinusoidal or the sum of sinusoids of frequency >2 Hz and having an average pressure value less than or equal to 2.5 cm H₂O in the vicinity of initial end 7.

In a preferred embodiment, the mean pressure value is between 0.75 and 1 cm H₂O and thus the peak-to-peak pressure value is between 1.5 and 2 cm H₂O.

In one embodiment, movement of fan 4 is only activated when the individual shows breathing activity.

Vibrations caused by rotation of the fan can cause discomfort for the patient. To overcome this problem, in a preferred embodiment of the present invention, during an initial stage of fan operation, called the acclimatisation stage, the rotation speed of fan 4 is controlled to produce a pressure stimulus whose peak-to-peak amplitude increases linearly to a predefined target value.

In another embodiment, during the acclimatisation stage, the rotation speed of fan 4 is controlled to produce a pressure stimulus whose peak-to-peak amplitude increases exponentially until it reaches a predefined target value, e.g. 2 cm H₂O.

In a further embodiment, during the acclimatisation stage, the rotation speed of fan 4 is controlled to produce a pressure stimulus whose peak-to-peak amplitude increases according to a curve of predefined shape until it reaches a predefined target value, e.g. 2 cm H₂O.

According to yet another embodiment, during the measurement stage the rotation speed of fan 4 is modulated by a closed-loop control system to produce a pressure stimulus whose ideal peak-to-peak amplitude is kept constant and equal to a pre-set value or one determined on the basis of one or more of the patient's pressure, flow, impedance, resistance and reactance values measured during the acclimatisation stage.

In another embodiment, during the measurement stage the rotation speed of fan 4 is modulated according to a succession of values to produce a pressure stimulus whose peak-to-peak amplitude is equal to a pre-set value or is determined on the basis of the patient's impedance, resistance and reactance values measured during the acclimatisation stage.

In another embodiment, during the measurement stage, the rotation speed of fan 4 is controlled to produce a pressure stimulus that generates flow oscillations of not less than a predefined value or is determined on the basis of the patient's impedance, resistance and reactance values measured during the acclimatisation stage.

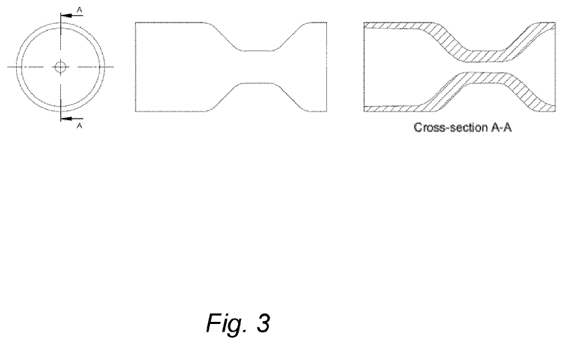

One problem which may arise when using the system according to the present invention is that of maintaining correct calibration of the pressure and flow sensors for impedance measurement. FIG. 3 shows a test device, of a shape consisting of 2 cones connected by one or more cylindrical ducts. By dimensioning the area of the cylindrical duct and its length it is possible to produce test objects characterised by time-stable mechanical impedance values which can be used for both automatically checking the calibration status of the sensors in the system according to the present invention and possibly calibrating them. The test device may be supplied in a kit together with the system according to the present invention, to allow the end user to carry out a calibration check on the system and, if necessary, calibrate it.

In another embodiment, respiratory chamber 2 and fan 4 may be removed and replaced. In order to allow the patient to breathe spontaneously through the circuit with minimum effort, the dimensions of respiratory chamber 2 and the air inlet and outlet areas near initial end 7 and terminal end 6 are preferably dimensioned to have a maximum impedance of 1 cm H₂O/L/s, measured at normal breathing frequencies and thus in the range 0-1 Hz.

A possible procedure for using the system described here to measure the mechanical impedance of the respiratory system is described below. When switched on, the system may ask the user to check for correct functioning using the test device supplied. The patient is then invited to breathe through the connection interface (filter, mouthpiece) connected to initial end 7. After identifying by means of the sensors that respiratory activity is present, the system will activate motor 3 and fan 4 and will initiate the acclimatisation stage during which impedance, resistance and reactance values are determined and continuously updated by the system on the basis of the pressure and flow values read by sensors 9 and 10. At the end of the acclimatisation stage the system will automatically switch to the measurement stage of predefined duration, at the end of which the measurement values will be stored.

The impedance of the respiratory system may be calculated using any impedance calculation algorithm such as, for example, an algorithm based on least-squares optimisation reported by Horowitz (Comput Biomed Res 1983 December; 16(6):499-521.) and Kackza (Ann Biomed Eng 1999 May; 27(3):340-55) and recently refined by Dellacà et al. (EP1551293). This algorithm is based on decomposing the pressure and flow signals into their components arising from normal respiratory activity and those arising from external stimulation. The latter are in turn decomposed into their constituent harmonics and an iterative calculation procedure is applied to each of them to identify the phase coefficients of each within time windows of predefined length W.

The phase coefficients of the pressure and flow signals determined for each of the harmonics (f) yield the impedance Z(f) relating to the data present within time window Z_{w}(f):

$${Z}_{w} ( f ) = \frac{{a}_{p} ( f ) + j * {b}_{p} ( f )}{{a}_{\dot{v}} ( f ) + j * {b}_{\dot{v}} ( f )} = {R}_{w} ( f ) + j * {X}_{w} ( f )$$

This calculation is then repeated, shifting the time window of one or more samples forward in order to obtain the calculation of Z(f) for all measured data.

Subsequently, the values of Z(f), R(f), X(f), pressure and flow are analysed using both thresholding and statistical outlier identification methods to identify and exclude portions of the data measured at measurement artefacts such as: glottis closure, swallowing, coughing, phonation, or leakage at the mouthpiece/filter used for measurement. Finally, the measured result is obtained by calculating one or more parameters derived from the Z(f), R(f), X(f), pressure and flow values relating to portions of free data not containing artefacts.

1. A portable system for measuring the mechanical impedance of an individual's respiratory system during spontaneous breathing, including:
a system for generating pressure signals that is able to produce small changes in pressure and/or flow at the opening to the airways, that includes a motor and a fan;
a detection system that is able to measure the values of pressure and flow produced by stimulation and by the individual's spontaneous breathing activity; and
a microprocessor that is able to:
control the system for generating pressure signals;
receive data measured by the detection system;
calculate a measure of the mechanical impedance of the individual's respiratory system on the basis of the values measured by the detection system;
characterised in that it comprises a first chamber that contains the motor and a second chamber that contains the fan, the first and second chambers being separate from each other so that there is no passage of air between the first and second chambers, the second chamber having a rear opening able to exchange air with the external environment and a front opening able to be brought into contact with the individual and to receive the individual's inspiratory and expiratory flows.

2. A portable system according to claim 1, in which the detection system comprises one or more of the following: at least one pressure sensor; at least one flow meter to measure the flow produced by the individual's spontaneous breathing activity.

3. A portable system according to claim 1, in which the flow produced by the individual's spontaneous breathing activity is estimated by the microprocessor on the basis of one or more of the following data: fan rotation speed, the motor's electricity consumption values, the pressure, temperature and humidity of the air.

4. A portable system according to claim 3, further characterised in that the microprocessor is arranged to modulate the pressure signals so that during an initial measurement stage the rotation speed is reduced in relation to a steady-state measurement stage.

5. A portable system according to claim 4, in which in the initial stage the rotation speed of the fan is controlled by the microprocessor to produce a pressure signal whose peak-to-peak amplitude increases linearly until a predefined target value is reached.

6. A portable system according to claim 4, in which in the initial stage the rotation speed of the fan is controlled by the microprocessor to produce a pressure signal whose peak-to-peak amplitude increases exponentially until reaching a predefined target value.

7. A portable system according to claim 4, in which during the steady-state measurement stage the rotation speed of the fan is controlled by a closed loop control system to produce a pressure signal whose peak-to-peak amplitude is kept constant and equal to a value determined on the basis of one or more of the individual's pressure, flow, impedance, resistance and reactance values measured during the initial measurement stage.

8. A method for operating a portable system as defined in claim 1, comprising the steps of:
generating pressure stimuli, producing pressure variations;
measuring the pressure and flow values produced by the stimulation and by the individual's spontaneous breathing activity;
receiving from the microprocessor the data measured by the detection system; and
calculating a measure of the mechanical impedance of the individual's respiratory system on the basis of the values measured by the detection system.

9. A computer program which, when executed by a microprocessor, implements the method according to claim 8.

10. A kit to measure the mechanical impedance of an individual's respiratory system during spontaneous breathing, comprising:
the system according to claim 1; and
a test and calibration device, comprising a hollow conduit with two substantially truncated conical opposite ends whose smaller cross-sections converge inwards, the two truncated conical ends being joined together by a substantially cylindrical central portion, the test device having a known and predetermined impedance value.

11. A portable system according to claim 2, in which the flow produced by the individual's spontaneous breathing activity is estimated by the microprocessor on the basis of one or more of the following data: fan rotation speed, the motor's electricity consumption values, the pressure, temperature and humidity of the air.

12. A portable system according to claim 2, further characterised in that the microprocessor is arranged to modulate the pressure signals so that during an initial measurement stage the rotation speed is reduced in relation to a steady-state measurement stage.

13. A portable system according to claim 5, in which during the steady-state measurement stage the rotation speed of the fan is controlled by a closed loop control system to produce a pressure signal whose peak-to-peak amplitude is kept constant and equal to a value determined on the basis of one or more of the individual's pressure, flow, impedance, resistance and reactance values measured during the initial measurement stage.

15. A portable system according to claim 6, in which during the steady-state measurement stage the rotation speed of the fan is controlled by a closed loop control system to produce a pressure signal whose peak-to-peak amplitude is kept constant and equal to a value determined on the basis of one or more of the individual's pressure, flow, impedance, resistance and reactance values measured during the initial measurement stage.

16. A method for operating a portable system as defined in claim 2, comprising the steps of:
generating pressure stimuli, producing pressure variations;
measuring the pressure and flow values produced by the stimulation and by the individual's spontaneous breathing activity;
receiving from the microprocessor the data measured by the detection system; and
calculating a measure of the mechanical impedance of the individual's respiratory system on the basis of the values measured by the detection system.

17. A method for operating a portable system as defined in claim 3, comprising the steps of:
generating pressure stimuli, producing pressure variations;
measuring the pressure and flow values produced by the stimulation and by the individual's spontaneous breathing activity;
receiving from the microprocessor the data measured by the detection system; and
calculating a measure of the mechanical impedance of the individual's respiratory system on the basis of the values measured by the detection system.

18. A method for operating a portable system as defined in claim 4, comprising the steps of:
generating pressure stimuli, producing pressure variations;
measuring the pressure and flow values produced by the stimulation and by the individual's spontaneous breathing activity;
receiving from the microprocessor the data measured by the detection system; and
calculating a measure of the mechanical impedance of the individual's respiratory system on the basis of the values measured by the detection system.

19. A method for operating a portable system as defined in claim 5, comprising the steps of:
generating pressure stimuli, producing pressure variations;
measuring the pressure and flow values produced by the stimulation and by the individual's spontaneous breathing activity;
receiving from the microprocessor the data measured by the detection system; and
calculating a measure of the mechanical impedance of the individual's respiratory system on the basis of the values measured by the detection system.

20. A method for operating a portable system as defined in claim 6, comprising the steps of:
generating pressure stimuli, producing pressure variations;
measuring the pressure and flow values produced by the stimulation and by the individual's spontaneous breathing activity;
receiving from the microprocessor the data measured by the detection system; and
calculating a measure of the mechanical impedance of the individual's respiratory system on the basis of the values measured by the detection system.
